(19) United States
(12) Patent Application Publication
(10) Pub. No.: US 2013/0317463 A1
(43) Pub. Date: Nov. 28, 2013

(54) NEGATIVE PRESSURE WOUND THERAPY SYSTEM WITH A BUFFERING UNIT

(71) Applicants: Nan-Kuang YAO, New Taipei City (TW); Luo-Hwa MIAU, New Taipei City (TW); Jhy-Wen WU, New Taipei City (TW); Jen-Chien CHIEN, New Taipei City (TW); Shi-Fu CHEN, New Taipei City (TW); Li-Ling LI, New Taipei City (TW)

(72) Inventors: Nan-Kuang YAO, New Taipei City (TW); Luo-Hwa MIAU, New Taipei City (TW); Jhy-Wen WU, New Taipei City (TW); Jen-Chien CHIEN, New Taipei City (TW); Shi-Fu CHEN, New Taipei City (TW); Li-Ling LI, New Taipei City (TW)

(73) Assignee: APEX MEDICAL CORP., New Taipei City (TW)

(21) Appl. No.: 13/477,393

(22) Filed: May 22, 2012

(51) Int. Cl. A61M 1/00

(52) U.S. Cl. 604/319

(57) ABSTRACT

A negative pressure wound therapy system has a buffering unit connected between an actuator and a wound dressing. The buffering unit has similar physical condition with the wound, and consumes its negative pressure to compensate for the negative pressure loss in the wound to resist the pressure rise in the wound during slight leakage. The buffering unit also keeps the actuator from directly operating on the wound to reduce the pain of the patient while lowering the pressure in the wound.

### BACKGROUND OF THE INVENTION

1. Field of the Invention

The present invention relates a negative pressure wound therapy system, especially to a negative pressure wound therapy system with a buffering unit.

2. Description of the Prior Arts

Negative pressure wound therapy utilizes wound sheets, soft suction pads, or biocompatible pore materials to attach on the wounds and connects to a vacuum pump. The vacuum pump creates negative pressure in the wound to extract the pus and infection subjects and to draw the healthy tissue fluid so that a moist therapy environment is maintained. Therefore, the blood circulation around the wound is promoted to accelerate wound healing. To provide negative pressure wound therapy, a conventional negative pressure wound therapy system is developed.

With reference to FIG. 6, when the conventional negative pressure wound therapy system is operated, the inner pressure of the collector 92 in front of the actuator 91 or the pressure at the detecting position 93 near the wound 94 is used as the feedback control pressure. The detecting position 93 is nearer the wound 94 than the collector 92 so that the pressure detected at the detecting position 93 is closer to the pressure in the wound 94. The controller 95 receives the signal from the sensor to actuate feedback control to determine whether the actuator 91 is restarted to maintain the negative pressure in the wound 94. Then the negative pressure in the wound 94 is kept in a certain range. However, the pressures at the detecting position 93 and in the collector 92 are both different from the pressure in the wound 94 so that the timing to restart the actuator 91 is inaccurate.

Additionally, the negative pressure in the wound undergoes slight changes frequently since a slight leakage occurs because a trace of pus and plasma may permeate out of the wound or the wound dress may be torn. If the controller restarts the actuator frequently because of the slightly changed negative pressure in the wound, or the actuator is kept operated at low speed when the negative pressure is steady, energy is consumed and the cost is increased. If the actuator is restarted when the true negative pressure in the wound is far from the desired negative pressure in the wound to recover the desired negative pressure, the rapid change of the pressure in the wound causes pain.

Moreover, one of the conventional negative pressure wound therapy systems has the actuator connected between the collector and the wound. The actuator directly operates on the wound so that the patient easily feels the pain when the actuator is operated.

To overcome the shortcomings, the present invention provides a negative pressure wound therapy system with a buffering unit to mitigate or obviate the aforementioned problems.

### SUMMARY OF THE INVENTION

The main objective of the present invention is to provide a negative pressure wound therapy system with a buffering unit to maintain the negative pressure in the wound without restarting the actuator. The negative pressure wound therapy system has a buffering unit connected between an actuator and a wound dressing. The buffering unit has the same physical condition with the wound, consumes its negative pressure to compensate for the negative pressure loss in the wound to resist the pressure rise in the wound during slight leakage. The buffering unit also keeps the actuator from directly operating on the wound to reduce the pain of the patient while lowering the pressure in the wound.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a negative pressure wound therapy system with a buffering unit in accordance with the present invention;

FIG. 2 is a block diagram of the negative pressure wound therapy in FIG. 1;

FIG. 3 is an exploded perspective view of the buffering unit of the negative pressure wound therapy system in FIG. 1;

FIG. 4 is a graph depicting time plotted against pressure of the negative pressure wound therapy system in FIG. 1, showing the pressure variation during the leaking and slight leakage;

FIG. 5 is a graph depicting time plotted against pressure of the negative pressure wound therapy system in FIG. 1, showing the pressure variation while alternatively opening the valve and restarting the actuator; and

FIG. 6 is a block diagram of a conventional negative pressure wound therapy system in accordance with the prior art.

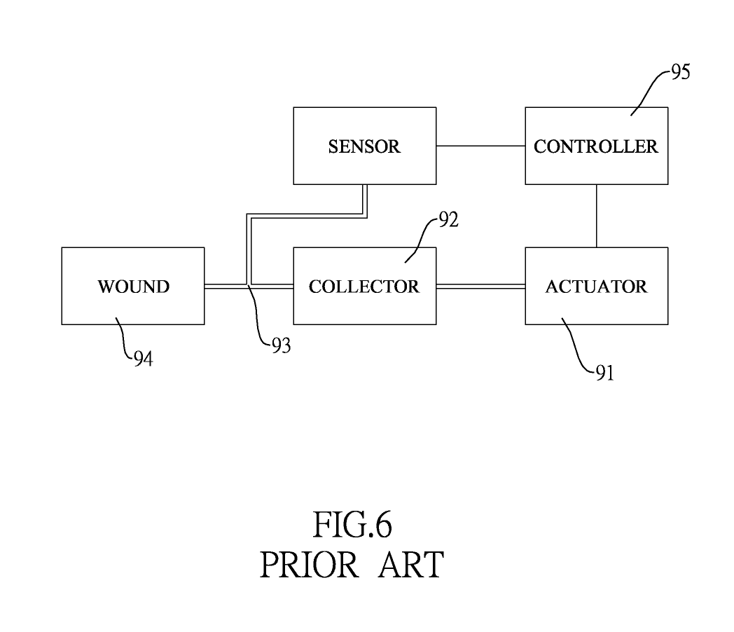

### DETAILED DESCRIPTION OF THE PREFERRED EMBODIMENTS

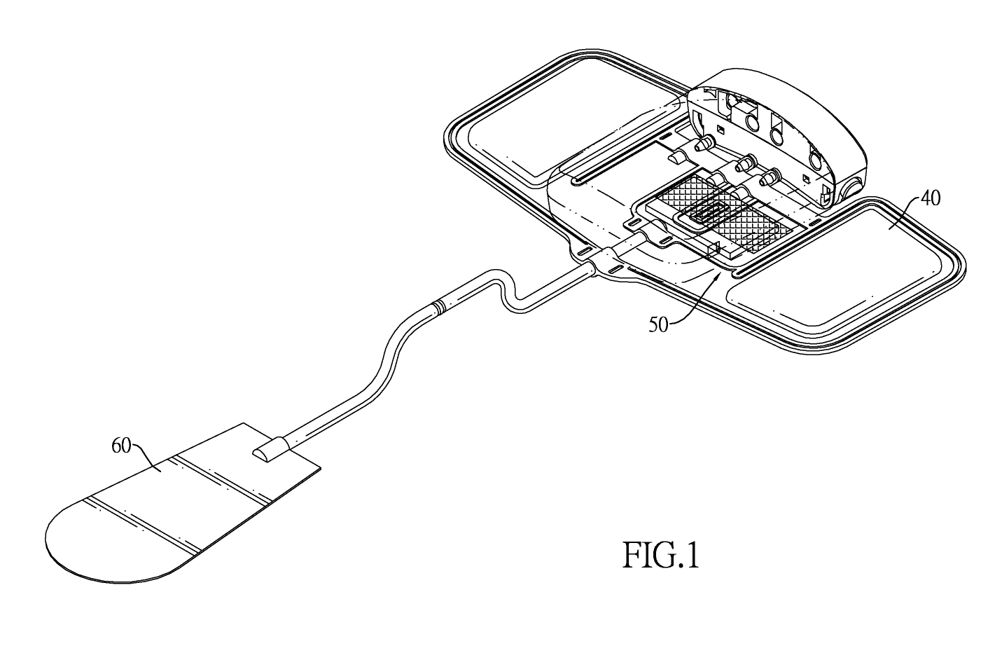

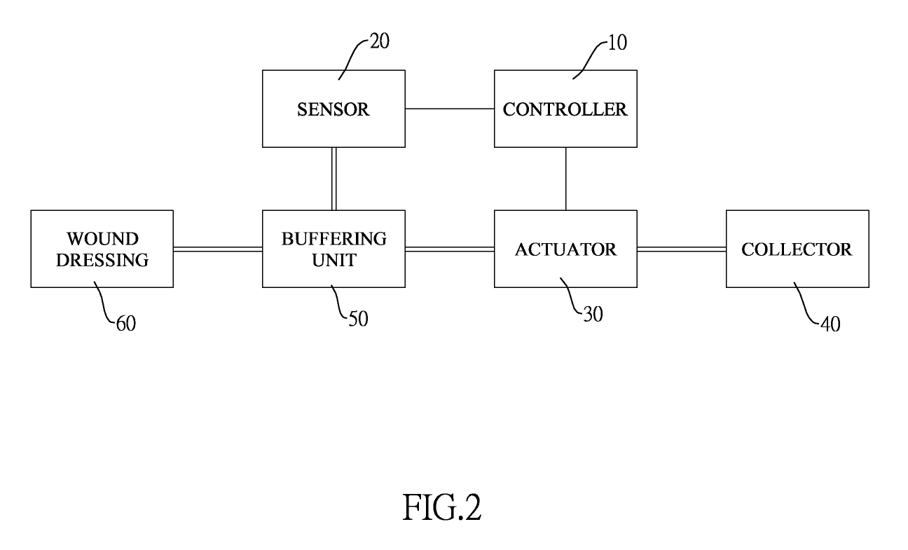

With reference to FIGS. 1 and 2, a negative pressure wound therapy system in accordance with the present invention comprises a controller 10, a sensor 20, an actuator 30, a collector 40, a buffering unit 50 and a wound dressing 60.

The controller 10 is electrically connected to the sensor 20 and the actuator 30. The sensor 20 sends signals to the controller 10, and then the controller 10 determines whether the actuator 30 is restarted. The actuator 30 is connected respectively to the buffering unit 50 and the collector 40. In a preferred embodiment, the actuator 30 is connected between the buffering unit 50 and the collector 40. The sensor 20 is connected to the buffering unit 50. The buffering unit 50 is connected between the wound dressing 60 and the actuator 30.

The buffering unit 50 has similar physical condition with the wound. Because the buffering unit 50 is connected to the wound dressing 60, the pressure at the buffering unit 50 is similar to the pressure in the wound. Therefore, the sensor 20 detects the pressure at the buffering unit 50 as a reference for feedback control. The buffering unit 50 consumes its negative pressure to compensate for the negative pressure loss in the wound dressing 60.

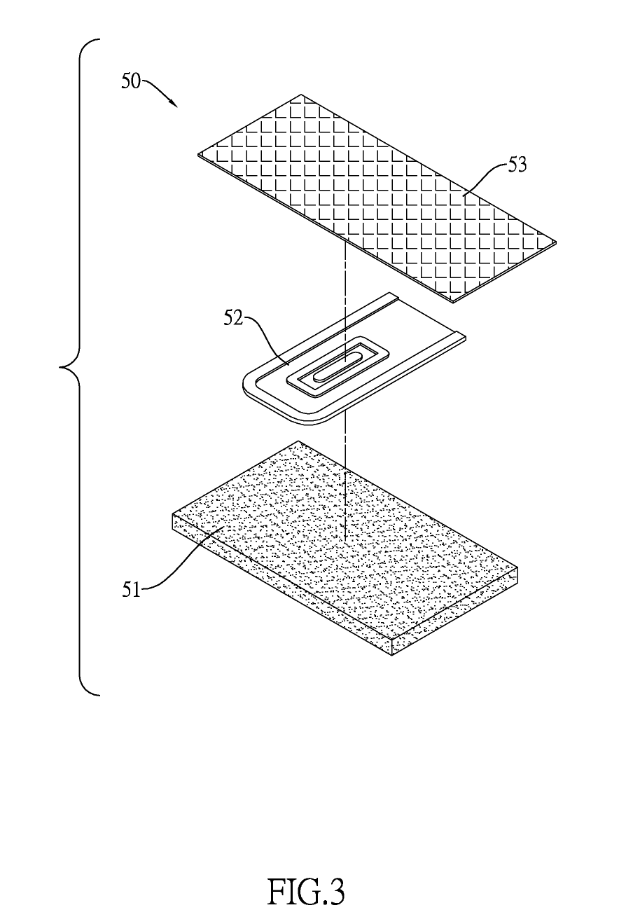

With reference to FIG. 3, in a preferred embodiment, the buffering unit 50 comprises a water-absorbing layer 51, an air-permeating layer 52 and a meshed layer 53. The buffering unit 50 may comprise one of the layers 51, 52, 53, or may comprise all of the layers 51, 52, 53. The air-permeating layer 52 is connected to the sensor 20. The water-absorbing layer 51 may be made of polymer foam material such as Polyvinyl Alcohol (PVA). The water-absorbing layer 51 has high water-absorbability and the volume of the water-absorbing layer 51 is easily expanded. The water-absorbing feature of the water-absorbing layer 51 is reversible. When the buffering unit 50 is in negative pressure, the volume of the water-absorbing layer 51 is compressed and the water-absorbing layer 51 drains out the water. When the buffering unit 50 comes off the negative pressure status, the volume of the water-absorbing layer 51 is expanded and performs sufficient water-absorption to absorb and save the remaining liquid in the tubes. The air-permeating layer 52 may be made of air-permeating but water-impermeable material such as Polyvinylidene fluoride (PVDF). The air-permeating layer 52 keeps the liquid from flowing into the sensor 20 and allows the air to flow into the sensor 20 so that the sensor 20 detects the negative pressure in the buffering unit 50 through the air-permeating layer 52. The meshed layer 53 may be made of thermoplastic resin such as Polypropylene (PP). The meshed layer 53 has meshes to simulate the wound tissue so that the liquid has appropriate flowing path to keep the buffering unit 50 from collapsing and pathological closing.

With reference to FIG. 2, when the negative pressure wound therapy system operates, the wound dressing 60 covers the patient's wound. The actuator 30 is started to create a negative pressure environment in the wound and to extract pus and infection subjects through the buffering unit 50. When the negative pressure reaches the predetermined value, the actuator 30 is stopped. The sensor 20 detects the negative pressure in the buffering unit 50 and sends feedback to the controller 10. The controller 10 depends on the detected negative pressure to determine whether the actuator 30 is restarted. The pus and infection subjects flows through the wound dressing 60, the buffering unit 50, and the actuator 30 in sequence, and then flows into the collector 40.

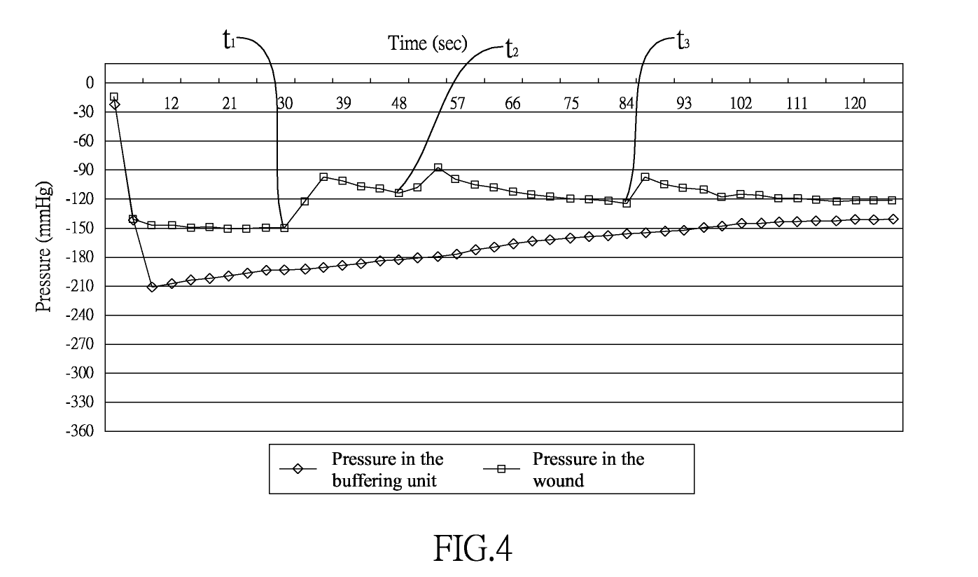

With reference to FIG. 4, the present invention creates leaking and slight leakage statuses to test the effect of the buffering unit. Initially, the actuator is started to make the pressure in the buffering unit to −210 mmHg and the pressure in the wound to −150 mmHg. Then the actuator is stopped.

At the time t₁, the system is leaked for a few seconds. The pressure in the wound is raised to almost −90 mmHg and then the system is stopped from leaking. The pressure of the buffering unit is raised up slowly to compensate for the negative pressure loss in the wound. The pressure in the wound is lowered to −120 mmHg and is not able to reach the initial negative pressure since the leaking status raises too much pressure. However, the buffering unit still compensates for some negative pressure loss in the wound. At the times t₂ and t₃, the system is slightly leaked for a few seconds. The pressure in the wound is raised to almost −90 mmHg and then the system is stopped from slightly leaking. The pressure of the buffering unit is raised up slowly to compensate for the negative pressure loss in the wound. The pressure in the wound is lowered to −120 mmHg to reach the pressure before the slight leakage occurs since the slight leakage makes less negative pressure loss. Therefore, the buffering unit effectively compensates for the negative pressure loss in the wound during slight leakage and the negative pressure in the wound quickly returns to the value before the slight leakage occurs.

With the aforementioned experiment, it is noted that the buffering unit immediately compensates for the negative pressure loss in the wound resulting from a trace of pus and plasma permeating out of the wound or the wound dress being torn without restarting the actuator. Therefore, the energy is saved since the actuator is not restarted frequently.

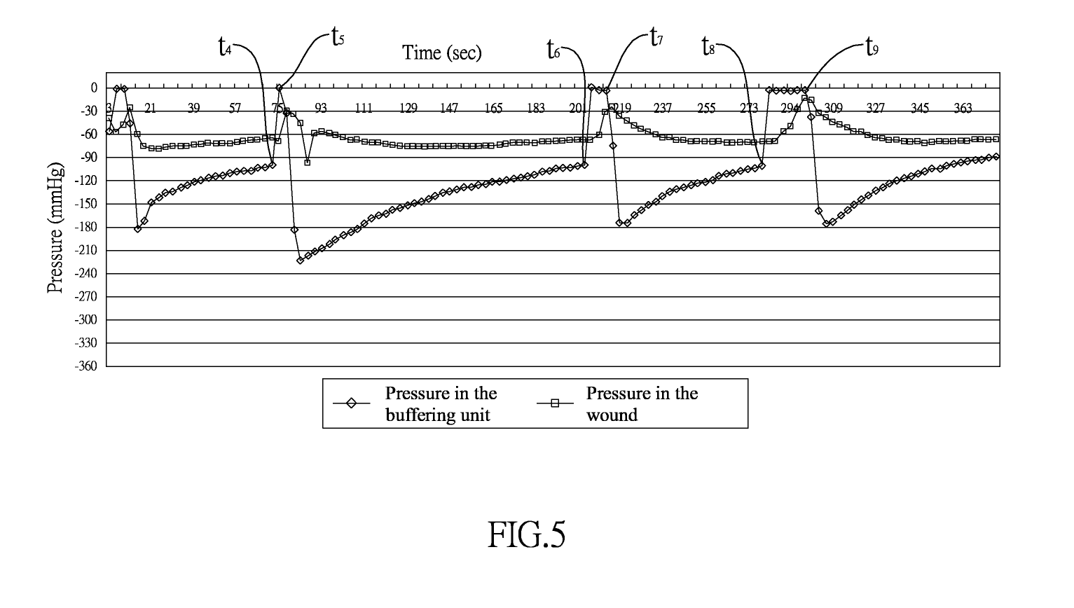

With reference to FIG. 5, the actuator is restarted manually and the valve is opened manually to create an active negative pressure environment. Initially, the actuator is started to make the negative pressure in the buffering unit reach −180 mmHg and the negative pressure in the wound reach −80 mmHg.

At the time t₄ and t₆, the valve is opened manually in a few seconds to raise the pressure in the buffering unit. In the same time, the pressure in the buffering unit rapidly raises from −100 mmHg to 0 mmHg but the pressure in the wound slowly raises from −70 mmHg to −30 mmHg. Therefore, it is noted that the rise of the pressure in the wound is less than the rise of the pressure in the buffering unit. At the time t₈, even the valve opening time is doubled, the pressure in the wound still rises slowly.

At the time t₅, t₇ and t₉, when the valve is closed, the actuator is restarted to lower the pressure. In the same time, the pressure in the buffering unit rapidly drops from 0 mmHg to −210 mmHg, but the pressure in the wound slowly drops from −30 mmHg to −60 mmHg and then keeps steady. Therefore, it is noted that the fall of the pressure in the wound is less than the fall of the pressure in the buffering unit.

With the aforementioned experiment, it is noted that since the actuator is directly operated on the buffering unit and is indirectly operated on the wound through the buffering unit, the buffering unit effectively buffers the impact to the wound while the actuator operates. Therefore, the pressure in the wound is raised and lowered in a gentle way, and the pain of the patient is reduced while the pressure is lowered.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A negative pressure wound therapy system comprising a controller, a sensor, an actuator, a collector, a wound dressing, and a buffering unit that responds to the negative pressure variation and compensates for the negative pressure loss in the wound, wherein
the controller is electrically connected to the sensor and the actuator;
the actuator is connected to the buffering unit and the collector;
the sensor is connected to the buffering unit; and
the buffering unit is connected between the wound dressing and the actuator.

2. The negative pressure wound therapy system as claimed in claim 1, wherein the buffering unit comprises a water-absorbing layer draining water while the volume of the water-absorbing layer is compressed and absorbing water while the volume of the water-absorbing layer is expanded.

3. The negative pressure wound therapy system as claimed in claim 1, wherein the buffering unit comprises an air-permeating layer connected to the sensor.

4. The negative pressure wound therapy system as claimed in claim 1, wherein the buffering unit comprises a meshed layer having meshes.

5. The negative pressure wound therapy system as claimed in claim 1, wherein the buffering unit comprises
a water-absorbing layer draining water while the volume of the water-absorbing layer is compressed and absorbing water while the volume of the water-absorbing layer is expanded;
an air-permeating layer connected to the sensor; and
a meshed layer having meshes.

6. The negative pressure wound therapy system as claimed in claim 2, wherein the water-absorbing layer is made of polymer foam material.

7. The negative pressure wound therapy system as claimed in claim 5, wherein the water-absorbing layer is made of polymer foam material.

8. The negative pressure wound therapy system as claimed in claim 4, wherein the meshed layer is made of thermoplastic resin.

9. The negative pressure wound therapy system as claimed in claim 5, wherein the meshed layer is made of thermoplastic resin.

10. The negative pressure wound therapy system as claimed in claim 4, wherein the meshed layer is made of Polypropylene.

11. The negative pressure wound therapy system as claimed in claim 5, wherein the meshed layer is made of Polypropylene.

12. The negative pressure wound therapy system as claimed in claim 3, wherein the air-permeating layer is made of air-permeating but water-impermeable material.

13. The negative pressure wound therapy system as claimed in claim 5, wherein the air-permeating layer is made of air-permeating but water-impermeable material.

14. The negative pressure wound therapy system as claimed in claim 3, wherein the air-permeating layer is made of Polyvinylidene fluoride.

15. The negative pressure wound therapy system as claimed in claim 5, wherein the air-permeating layer is made of Polyvinylidene fluoride.

16. The negative pressure wound therapy system as claimed in claim 1, wherein the actuator is connected between the buffering unit and the collector.

17. The negative pressure wound therapy system as claimed in claim 5, wherein the actuator is connected between the buffering unit and the collector.

18. The negative pressure wound therapy system as claimed in claim 7, wherein the actuator is connected between the buffering unit and the collector.

19. The negative pressure wound therapy system as claimed in claim 11, wherein the actuator is connected between the buffering unit and the collector.

20. The negative pressure wound therapy system as claimed in claim 15, wherein the actuator is connected between the buffering unit and the collector.
